(19) United States
(12) Patent Application Publication
(10) Pub. No.: US 2019/0117786 A1
(43) Pub. Date: Apr. 25, 2019

(54) SELF-ASSEMBLING VERTEPORFIN AMPHIPHILES (SAVA) FOR LOCAL CANCER THERAPY

(71) Applicant: THE JOHNS HOPKINS UNIVERSITY, Baltimore, MD (US)

(72) Inventors: Sagar Ramesh Shah, Clemson, SC (US); Juan Carlos Martinez-Gutierrez, Baltimore, MD (US); Alejandro Ruiz-Valls, Baltimore, MD (US); Ran Lin, Baltimore, MD (US); Honggang Cui, Lutherville, MD (US); Alfredo Quinones-Hinojosa, Bel Air, MD (US)

(21) Appl. No.: 16/068,933

(22) PCT Filed: Jan. 11, 2017
(86) PCT No.: PCT/US2017/012936

§ 371 Date: Jul. 10, 2018

Related U.S. Application Data
(60) Provisional application No. 62/277,102, filed on Jan. 11, 2016.

(51) Int. Cl. A61K 47/64; A61K 31/409; A61P 35/00; A61N 5/10

(52) U.S. Cl. CPC A61K 47/64; A61N 5/10; A61P 35/00; A61K 31/409

(57) ABSTRACT

The present invention provides compositions comprising Verteporfin and other anticancer compounds linked to a hydrophilic peptide through a degradable linker molecule to allow the anticancer compounds to penetrate tissues via in situ administration. The compounds of the present invention are useful for sensitizing tumor cells to radiotherapy, preventing recurrence of tumors after surgical resection and for treating remaining unremoved cancer cells at the site of the tumor.

### REFERENCE TO RELATED APPLICATIONS

This application claims the benefit of U.S. Provisional Patent Application No. 62/277,102, filed on Jan. 11, 2016, which is hereby incorporated by reference for all purposes as if fully set forth herein.

### STATEMENT OF GOVERNMENTAL INTEREST

This invention was made with government support under grant nos. R01NS070024 from the National Institutes of Health, and DMR1255281 from the National Science Foundation. The government has certain rights in the invention.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Dec. 29, 2015, is named P13797-01_ST25.txt and is 399 bytes in size.

### BACKGROUND OF THE INVENTION

Therefore, there still exists a need for treatment of cancer in situ while limiting proliferation and metastasis without systemic treatment with anticancer agents.

### SUMMARY OF THE INVENTION

The present invention presents the further development of a new class of self-assembling amphiphile drugs useful in the treatment of disease. The present invention provides self-assembling verteporfin amphiphiles (SAVA) that can be locally administered in-situ, into a location in the body of a subject. In some embodiments, these SAVA can be administered into the site of tumor or tumor resection sites for effective cancer treatments. These SAVA can spontaneously associate into self-supporting gels in aqueous conditions, such as cell media, body fluids, and tissue. SAVA are easy-to-manufacture supramolecular hydrogels that will remain in the delivered site, and gradually release the anticancer drug, Verteporfin, over an extended period of time at a constant rate.

The data disclosed herein shows that Verteporfin targets potent oncogenes including, but not limited to YAP and TEAD in multiple cancers. This technology presents a new platform for improved treatment of tumors due to its potent effect on suppressing cell proliferation, stemness, migration, invasion/metastasis, metabolism, radiation resistance, and tumor growth; potentially extending patient survival.

In accordance with an embodiment, the present invention provides a self-assembling verteporfin amphiphile composition (SAVA) having the formula: V-Pep; wherein V-Pep comprises at least one or more verteporfin molecules (V) conjugated to a hydrophilic peptide composition (Pep); wherein Pep is a hydrophilic peptide composition having the amino acid sequence L-Bₙ-(T)_{z}, L is an C₂-C₆ alkyl linker having at least one or more disulfide bonds; Bₙ is an amino acid linker, of n=0 to 12 amino acids, which can be the same or different, and; T is a targeting peptide of z=1 to 15 peptides, with biologically relevant properties including, but not limited to, tumor binding, tissue penetrating peptides, cell penetrating peptides, apoptotic peptides) or capable of binding to known cellular epitopes, such as integrins or cancer cell receptors.

In accordance with an embodiment, the present invention provides a self-assembling verteporfin amphiphile composition (SAVA) having the formula of formula I:

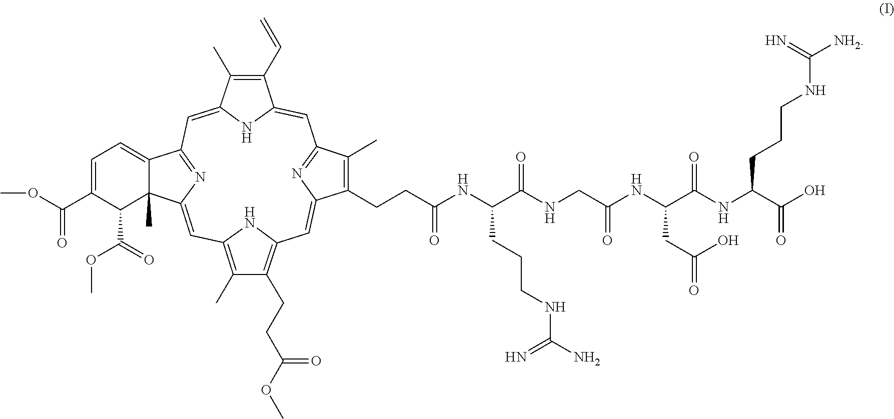 [SMILES: C=Cc1c(C)c2cc3nc(cc4[nH]c(cc5nc(cc1[nH]2)C(C)=C5CCC(=O)N[C@@H](CCCNC(=N)N)C(=O)NCC(=O)N[C@@H](CC(=O)O)C(=O)N[C@@H](CCCNC(=N)N)C(=O)O)c(CCC(=O)OC)c4C)[C@@]1(C)C3=CC=C(C(=O)OC)[C@H]1C(=O)OC]

In accordance with another embodiment, the present invention provides a self-assembling verteporfin amphiphile composition (SAVA) having the formula of formula II:

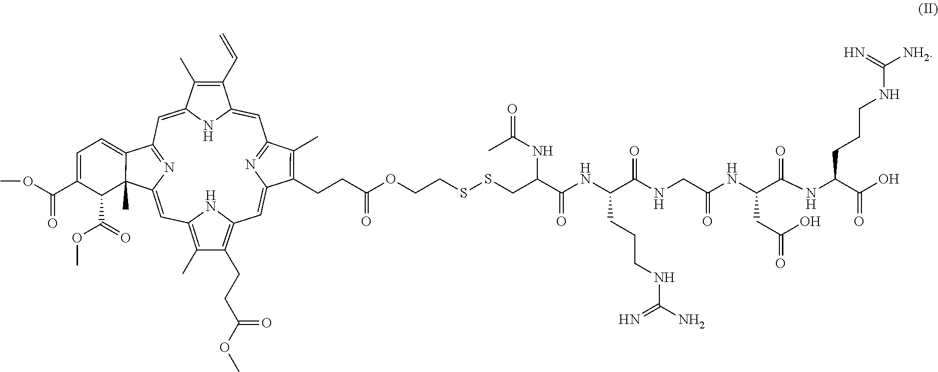 [SMILES: C=Cc1c(C)c2cc3nc(cc4[nH]c(cc5nc(cc1[nH]2)C(C)=C5CCC(=O)OCCSSCC(NC(C)=O)C(=O)N[C@@H](CCCNC(=N)N)C(=O)NCC(=O)N[C@@H](CC(=O)O)C(=O)N[C@@H](CCCNC(=N)N)C(=O)O)c(CCC(=O)OC)c4C)[C@@]1(C)C3=CC=C(C(=O)OC)[C@H]1C(=O)OC]

In accordance with a further embodiment, the present invention provides a SAVA composition comprising the compositions described above, and at least one biologically active agent (D) in a mixture.

In accordance with an embodiment, the present invention provides a method for treating a tumor in a subject comprising administering to the subject at the site of the tumor, an effective amount of the compositions described above.

In accordance with another embodiment, the present invention provides a method for treating a tumor in a subject comprising administering to the subject at the site of the tumor, an effective amount of the compositions described above, and at least one biologically active agent (D) in a mixture.

In accordance with another embodiment, the present invention provides a method of treating cancer in a subject comprising a) administering to the subject an effective amount of the making the SAVA compositions of the present invention, and a pharmaceutically acceptable carrier, in one or more doses, and b) administering ionizing radiation to the subject in proximity to the location of the cancer in the subject.

### BRIEF DESCRIPTION OF THE DRAWINGS

FIG. 1 illustrates an embodiment of the SAVA compositions of the present invention. The illustration depicts the self-assembly of amphiphilic monomers into nanofiber hydrogels is depicted. These nanofiber structures enmesh to form hydrogels under certain physiological conditions. The low viscosity of the monomer and nanofiber states offers great flexibility in handling, processing, and delivery. In an embodiment, the anticancer drug depicted can be verteporfin, and the linker can be a lower alkyl having a disulfide linkage to the peptide.

FIGS. 2A-2B show graphs depicting RP-HPLC trace (A) and ESI MS (B) profile of Ver-RGDR showing high purity and the expected molecular mass.

FIG. 3 is a pair of TEM images where Ver-RGDR was dissolved in DI water at 5 mM. After 24 aging, PBS buffer was added to trigger hydrogel formation. Short fibrous structures were observed in TEM images.

FIGS. 4A-4C show that verteporfin treatment with an embodiment of the SAVA compositions of the present invention decreases proliferation of non-CNS tumors. (4a) Proliferation of non-CNS tumors assessed after treatment with verteporfin (12.5 μM) for 5 days using MTT. (4b) Proliferation of non-CNS tumors assessed after treatment with verteporfin (12.5 μM) for 6 days using MTT. (4c) Origin/classification of cell lines tested in A and B.

FIGS. 5A-5B show that that verteporfin treatment with an embodiment of the SAVA compositions of the present invention radiosensitizes tumors. Pretreatment of KT21G1 meningioma cell line with VP SAVA composition for 12 hours resulted in increased radiation induced apoptosis at (5a) increasing doses of radiation and (5b) in a time-dependent manner. Comparisons to vehicle or VP alone as controls. Origin and classification of cell lines tested in 5a and 5b.

FIGS. 6A-6B depict verteporfin treatment with an embodiment of the SAVA compositions of the present invention decreases cell survival in malignant meningioma cell line KT21G1. Verteporfin decreases cell survival in malignant meningiomas in vitro at 3 days (6A) and 5 days (6B) of treatment; assessed using MTT. Colored #=significant versus Control cells; P<0.05, Student's t-test.

FIGS. 7A-7B depict verteporfin treatment with an embodiment of the SAVA compositions of the present invention decreases cell survival in malignant meningioma cell line IOMM-Lee. Verteporfin decreases cell survival in malignant meningiomas in vitro at 3 days (7A) and 5 days (7B) of treatment; assessed using MTT. Colored #=significant versus Control cells; P<0.05, Student's t-test.

FIGS. 8A-8C show verteporfin treatment with an embodiment of the SAVA compositions of the present invention has a profound dose dependent effect on glioblastoma proliferation and cell survival. verteporfin SAVA compositions were tested in two primary patient-derived GBM cell lines, JHGB612 (8A) and GBM1A (8B). (8C) verteporfin has a dose dependent effect on chordoma cell survival and proliferation using a patient-derived primary chordoma cell line, JHC7. Please note that (a, b, c) were all assessed using MTT assay. Colored #=significant versus Control cells; P<0.05, Student's t-test.

### DETAILED DESCRIPTION OF THE INVENTION

In accordance with some embodiments the invention provides a new application and novel assembling of the drug, verteporfin, using a local delivery platform that allows controlled release of verteporfin-amphiphiles locally at the site of a tumor. Without being limited to any particular theory, the present inventors have found that verteporfin targets potent oncogenes including, but not limited to YAP and TEAD in multiple cancers. This self-assembling verteporfin composition becomes a hydrogel when introduced into the tissues of a subject, and the hydrogel formulation increases delivery of the drug directly to the tumor bed and surrounding parenchyma containing infiltrative tumor cells that cannot be surgically resected or identified using current imaging modalities. Given its liquid presentation and gel formation upon contact with human tissue at the site of resection, the inventive compositions can be delivered with widely available and universally-adopted clinical tools, including but not limited to standard syringe/needle applicators. Hence, no significant training is required by the medical personnel and/or surgeons for its administration. By administering the inventive compositions in a liquid-based package into the tumor or tumor resection site, the inventive compositions and methods present a simple and feasible adjuvant to the current standard of care of cancer patients at the time of surgery. Currently, the adverse effects of systemic verteporfin has limited its use to single dose applications. However, the use of the localized verteporfin delivery system of the present invention eliminates such adverse effects and will allow wider use of this drug.

The present invention provides herein the design of new monodisperse, amphiphilic prodrugs of verteporfin (SAVA) that can spontaneously associate into discrete, stable hydrogels with supramolecular nanostructures. These nanofiber hydrogels follow similar principles as those first developed in International Patent Publication No. WO 2014/066002, and incorporated by reference herein. The very nature of the molecular design ensures that a fixed and tunable drug loading can be achieved, without the use of any additional carriers or matrices. The use of these amphiphilic prodrugs for local treatment of diseases and conditions such as cancer.

In order to imbue these properties upon a drug or biologically active agent, such as, for example, verteporfin, for cancer-related diseases, a peptide or oligopeptide with overall hydrophilicity (Pep) is biodegradably linked with the drug or biologically active agent. The peptide or oligopeptide chosen increases the aqueous solubility of the drug or biologically active agent and can promote the formation of well-defined one-dimensional nanostructure architectures including, but not limited to, cylindrical micelles, hollow nanotubes, filaments, fibrils, twisted ribbons, helical ribbons, nanobelts, nanofibers, through preferred secondary structure formation, e.g. beta sheet, alpha helix, poly proline type-II helix, and beta turns. In some embodiments, the SAVA compositions of the present invention are capable of forming three dimensional nanofiber networks and hydrogels in aqueous conditions.

As such, the SAVA compositions of the present invention form nanofiber hydrogels that can provide a sustained release local drug delivery system.

In accordance with an embodiment, the present invention provides a self-assembling verteporfin amphiphile composition (SAVA) having the formula: V-Pep; wherein V-Pep comprises at least one or more verteporfin molecules (V) conjugated to a hydrophilic peptide composition (Pep).

In some embodiments, Pep is a hydrophilic peptide composition having the amino acid sequence L-Bₙ-(T)_{z}, L is an C₂-C₆ alkyl linker having at least one disulfide bond; Bₙ is an amino acid linker, of n=0 to 12 amino acids, which can be the same or different, and; T is a targeting peptide of z=1 to 15 amino acids, with biologically relevant properties including, but not limited to, tumor binding, tissue penetrating peptides, cell penetrating peptides, apoptotic peptides) or capable of binding to known cellular epitopes, such as integrins or cancer cell receptors.

As used herein, the term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups.

Moreover, the term “alkyl” (or “lower alkyl”) includes both “unsubstituted alkyls” and “substituted alkyls,” the latter of which refers to alkyl moieties having substituents replacing hydrogen on one or more carbons of the hydrocarbon backbone. Such substituents may include, for example, a halogen, a hydroxyl, a carbonyl (such as a carboxyl, an alkoxycarbonyl, a formyl or an acyl), a thiocarbonyl (such as a thioester, a thioacetate, or a thioformate), an alkoxyl, a phosphoryl, a phosphonate, a phosphinate, an amino, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls may be further substituted with alkyls, alkenyls, alkoxys, alkylthios, aminoalkyls, carbonyl-substituted alkyls, —CF₃, —CN and the like.

In accordance with one or more embodiments, V can be conjugated to Pep (V-Pep) through the use of a chemical linker molecule L. The linker can be a sulfide bond, ester bond, amide bond, carbonate bond, hydrozone, or any amino acid with a side chain having a free amino, carboxyl or thiol group, or a short peptide that can be specifically cleaved by a particular enzyme or proteinase.

In accordance with an embodiment, the present invention provides a method of local administration of one or more biologically active agents to a subject comprising in situ application of a SAVA composition to the site of a tumor in a subject.

In accordance with an embodiment, the present invention provides a method of local administration of one or more biologically active agents to a subject comprising in situ injection of a SAVA composition, and upon contact with body fluids the composition is capable of undergoing a change from solution state to nanofiber gelation state.

In accordance with an embodiment, the delivered SAVA compositions of the present invention can sustainably release the encapsulated bioactive agents over a long period of time.

In accordance with an embodiment, the released bioactive agent can exert effective in vitro efficacy in killing a number of cancer cell lines and primary cells derived from human patients.

In accordance with an embodiment, the SAVA compositions of the present invention contain a fixed loading of the biological agents which is tunable and precisely defined by the molecular design, and will not require additional matrices/hydrogels for the delivery of the biological agents.

In accordance with an embodiment, the nanofiber form enables diffusion across larger areas relative to individual molecules and avoids capillary loss.

In accordance with an embodiment, the chemical conjugation of biological agents to a short peptide offers an efficient strategy to overcome the Multidrug resistance (MDR) mechanisms that cancer cells possess or may develop over the course of the treatment.

It is contemplated that the SAVA compositions of the present invention can be made in solid, or liquid form, and then applied to the tissues of interest by spraying, injection, or otherwise applying the compositions directly to the tissues.

In some preferred embodiments, the compositions of the present invention are prepared as a dry powder and then come in contact with aqueous solutions, for example, such as physiological buffers or tissue fluids such as blood or lymph, and will spontaneously form aqueous nanofiber hydrogels. In alternative embodiments, the compositions of the present invention can be formulated in a viscous liquid or vitrigel form and then are applied to the tissues of interest to become aqueous nanofiber hydrogels.

In some embodiments, the biologically active agent or drug, such as verteporfin (V) acts as the hydrophobic portion of molecule in the nanofiber hydrogel compositions of the present invention.

It is contemplated that the other hydrophobic molecules can be used in the SAVA compositions of the present invention. For example, other hydrophobic molecules such as steroids, other conjugated ring containing molecules, and hydrophobic drugs can be used.

As used herein, the term “hydrophobic” biologically active agents or drug molecules (D) describes a heterogeneous group of molecules that exhibit poor solubility in water but that are typically, but certainly not always, soluble in various organic solvents. Often, the terms slightly soluble (1-10 mg/ml), very slightly soluble (0.1-1 mg/ml), and practically insoluble (<0.1 mg/ml) are used to categorize such substances. Drugs such as steroids and many anticancer drugs are important classes of poorly water-soluble drugs; however, their water solubility varies over at least two orders of magnitudes. Typically, such molecules require secondary solubilizers such as carrier molecules, liposomes, polymers, or macrocyclic molecules such as cyclodextrins to help the hydrophobic drug molecules dissolve in aqueous solutions necessary for drug delivery in vivo. Other types of hydrophobic drugs show even a lower aqueous solubility of only a few ng/ml. Since insufficient solubility commonly accompanies undesired pharmacokinetic properties, the high-throughput screening of kinetic and thermodynamic solubility as well as the prediction of solubility is of major importance in discovery (lead identification and optimization) and development.

In some embodiments, Pep is a peptide composition having the amino acid sequence L-Bₙ-(T)_{z}, wherein L is an C₂-C₆ alkyl linker having at least one or more disulfide bonds; Bₙ is an amino acid, of n=0 to 12 amino acids, which can be the same or different, and T is a peptide of z=1 to 15 peptides, with biologically relevant properties including, but not limited to, tumor targeting, tissue penetrating, cell penetrating, apoptotic) or capable of binding to known cellular epitopes, such as integrins or cancer cell receptors.

In accordance with one or more embodiments, V and/or D can be conjugated to Pep through the use of a chemical linker. The linker can be a disulfide bond, ester bond (which can be cleaved by hydrolysis), amide bond, carbonate bond, hydrozone linker (which can be cleaved in low pH), or any amino acid with a side chain having a free amino, carboxyl or thiol group, or a short peptide that can be specifically cleaved by a particular enzyme or proteinase, for example GFLG or valine-citrulline linker (cleavable with enzyme cathepsin B).

In some embodiments L is an alkyl linker, where the linker has a sulfhydryl group and a hydroxyl, carbonyl or other reactive functional group. For example, L can be a molecule such a 2-mercaptoethanol. Disulfide bonds in the linker allow the drug, such as verteporfin, to be released from the peptide moiety by enzymatic cleavage. In some embodiments, the enzyme cleavage is through the enzyme, glutathione, in the tissues of the subject.

In some embodiments, T can be RGD or RGDR (SEQ ID NO: 1) or HDK or derivatives thereof, having z=1 to 6 repeating moieties.

Other possible targeting peptides which can be used in conjunction with the compositions of the present invention include tumor associated antigens. Examples of such antigents include CEA, TAG-72, CyclinB1, Ep-CAM, Her2/neu, CDK4, fibronectin, p53, ras, and other.

As used herein, the term “biologically active agent” include any compound, biologics for treating cancer-related diseases, e.g. drugs, inhibitors, and proteins. An active agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc.

Specific examples of useful biologically active agents the above categories include: anti-neoplastics such as androgen inhibitors, antimetabolites, cytotoxic agents, and immunomodulators. More specifically, non-limiting examples of useful biologically active agents include the following therapeutic categories antineoplastic agents, such as alkylating agents, nitrogen mustard alkylating agents, nitrosourea alkylating agents, antimetabolites, purine analog antimetabolites, pyrimidine analog antimetabolites, hormonal antineoplastics, natural antineoplastics, antibiotic natural antineoplastics, and vinca alkaloid natural antineoplastics, such as carboplatin and cisplatin; carmustine (BCNU); methotrexate; fluorouracil (5-FU) and gemcitabine; goserelin, leuprolide, and tamoxifen, aldesleukin, interleukin-2, docetaxel, etoposide, interferon; paclitaxel, other taxane derivatives, tretinoin (ATRA); bleomycin, dactinomycin, daunorubicin, doxorubicin, and mitomycin; vinblastine and vincristine.

In accordance with some embodiments, the biologically active agents (D) covalently linked to Pep include verteporfrin, vorapaxar, camptothecin, bumetanide and paclitaxel.

In accordance with another embodiment, the present invention provides a SAVA composition having the following formula:

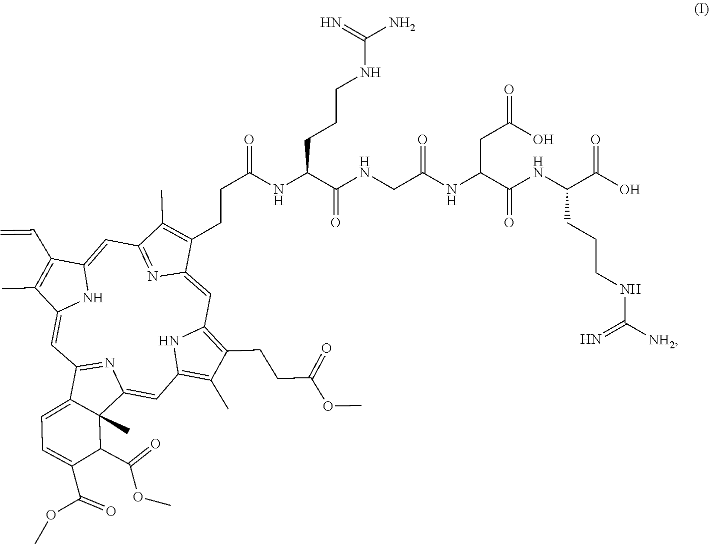

wherein V is the drug verteporfin, Pep comprises L₀, (B)₀ and T is (RGDR) (SEQ ID NO: 1) with z=1, and wherein the verteporfin molecule is covalently linked via a lysine amino acid linker.

In accordance with an embodiment the present invention provides a SAVA composition having the following formula:

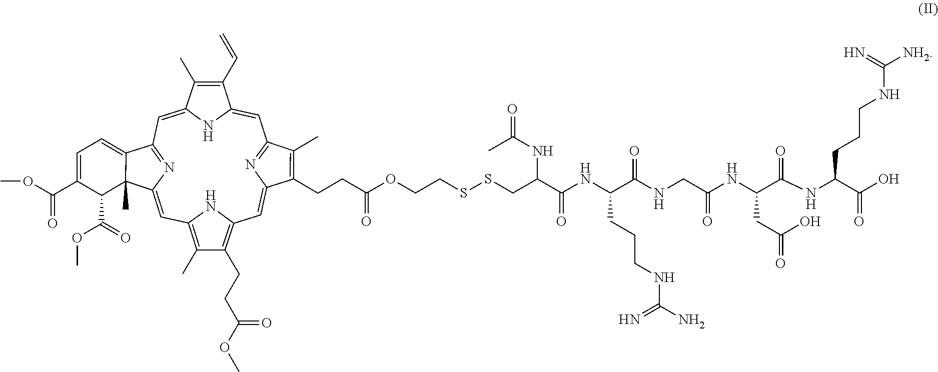

Wherein V is the drug verteporfin, Pep comprises a linker L of 2-mercaptoethanol, B₁ is cysteine and T is (RGDR) (SEQ ID NO: 1) with z=1, and wherein the verteporfin molecule is covalently linked via the linker to the peptide.

As used herein, the term “biologically active agent” (D) can also include imaging agents for use in identifying the location of the molecules in the tissues. In accordance with an embodiment, the imaging agent is a fluorescent dye. The dyes may be emitters in the visible or near-infrared (NIR) spectrum. Known dyes useful in the present invention include carbocyanine, indocarbocyanine, oxacarbocyanine, thüicarbocyanine and merocyanine, polymethine, coumarine, rhodamine, xanthene, fluorescein, borondipyrromethane (BODIPY), Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800RS, IRDye 700DX, ADS780WS, ADS830WS, and ADS832WS.

Organic dyes which are active in the NIR region are known in biomedical applications. However, there are only a few NIR dyes that are readily available due to the limitations of conventional dyes, such as poor hydrophilicity and photostability, low quantum yield, insufficient stability and low detection sensitivity in biological system, etc. Significant progress has been made on the recent development of NIR dyes (including cyanine dyes, squaraine, phthalocyanines, porphyrin derivatives and BODIPY (borondipyrromethane) analogues) with much improved chemical and photostability, high fluorescence intensity and long fluorescent life. Examples of NIR dyes include cyanine dyes (also called as polymethine cyanine dyes) are small organic molecules with two aromatic nitrogen-containing heterocycles linked by a polymethine bridge and include Cy5, Cy5.5, Cy7 and their derivatives. Squaraines (often called Squarylium dyes) consist of an oxocyclobutenolate core with aromatic or heterocyclic components at both ends of the molecules, an example is KSQ-4-H. Phthalocyanines, are two-dimensional 18π-electron aromatic porphyrin derivatives, consisting of four bridged pyrrole subunits linked together through nitrogen atoms. BODIPY (borondipyrromethane) dyes have a general structure of 4,4′-difluoro-4-bora-3a, 4a-diaza-s-indacene) and sharp fluorescence with high quantum yield and excellent thermal and photochemical stability.

Other imaging agents which are attached to the SAVA compositions of the present invention include PET and SPECT imaging agents. The most widely used agents include branched chelating agents such as di-ethylene tri-amine penta-acetic acid (DTPA), 1,4,7,10-tetra-azacyclododecane-1,4,7,10-tetraacetic acid (DOTA) and their analogs. Chelating agents, such as di-amine dithiols, activated mercaptoacetyl-glycyl-glycyl-gylcine (MAG3), and hydrazidonicotinamide (HYNIC), are able to chelate metals like ^{99m}Tc and ¹⁸⁶Re. Instead of using chelating agents, a prosthetic group such as N-succinimidyl-4-¹⁸F-fluorobenzoate (¹⁸F-SFB) is necessary for labeling peptides with ¹⁸F. In accordance with a preferred embodiment, the chelating agent is DOTA.

Various forms of the biologically active agents may be used. These include, without limitation, such forms as uncharged molecules, molecular complexes, salts, ethers, esters, amides, prodrug forms and the like, which are biologically activated when implanted, injected or otherwise placed into a subject.

In some embodiments, the linker can be any amino acid with a side chain having a free amino, carboxyl or disulfide group. Exemplary amino acids useful as amino acid linkers in the SAVA compositions of the present invention include lysine (K), glutamic acid (E), arginine (R) and cysteine (C).

It is contemplated that verteporfin (V) and/or other biologically active agents (D) are covalently linked to the Pep via a biodegradable bond. For example, amino groups, carboxyl groups and disulfide bonds are capable of being cleaved in vitro by various chemical and biological or enzymatic processes.

In certain embodiments, S compositions of the present invention biodegrade within a period that is acceptable in the desired application. In certain embodiments, such as in vivo therapy, such degradation occurs in a period usually less than about five years, one year, six months, three months, one month, fifteen days, five days, three days, or even one day on exposure to a physiological solution with a pH between 6 and 8 having a temperature of between about 25 and 37° C. In other embodiments, the nanofiber hydrogel degrades in a period of between about one hour and several weeks, depending on the desired application. In some embodiments, the SAVA compositions may include a detectable agent that is released on degradation.

“Gel” refers to a state of matter between liquid and solid, and is generally defined as a cross-linked polymer network swollen in a liquid medium. Typically, a gel is a two-phase colloidal dispersion containing both solid and liquid, wherein the amount of solid is greater than that in the two-phase colloidal dispersion referred to as a “sol.” As such, a “gel” has some of the properties of a liquid (i.e., the shape is resilient and deformable) and some of the properties of a solid (i.e., the shape is discrete enough to maintain three dimensions on a two-dimensional surface).

By “hydrogel” is meant a water-swellable polymeric matrix that can absorb water to form elastic gels, wherein “matrices” are three-dimensional networks of macromolecules held together by covalent or noncovalent crosslinks. On placement in an aqueous environment, dry hydrogels swell by the acquisition of liquid therein to the extent allowed by the degree of cross-linking.

Starting materials and reagents used in preparing these nanofiber hydrogel compositions of the present invention are either available from commercial suppliers such as Aldrich Chemical Company (Milwaukee, Wis.), Bachem (Torrance, Calif.), Sigma (St. Louis, Mo.), or are prepared by methods well known to the person of ordinary skill in the art following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supplements, Elsevier Science Publishers, 1989; Organic Reactions, vols. 1-40, John Wiley and Sons, New York, N.Y., 1991; March J; Advanced Organic Chemistry, 4^{th} ed. John Wiley and Sons, New York, N.Y., 1992; and Larock: Comprehensive Organic Transformations, VCH Publishers, 1989. In most instances, amino acids and their esters or amides, and protected amino acids, are widely commercially available; and the preparation of modified amino acids and their amides or esters are extensively described in the chemical and biochemical literature and thus well-known to persons of ordinary skill in the art. For example, N-pyrrolidineacetic acid is described in Dega-Szafran Z and Pryzbylak R. Synthesis, IR, and NMR studies of zwitterionic α-(1-pyrrolidine)alkanocarboxylic acids and their N-methyl derivatives. J. Mol. Struct.: 436-7, 107-121, 1997; and N-piperidineacetic acid is described in Matsuda O, Ito S, and Sekiya M. each article herein expressly incorporated herein fully by reference.

Conveniently, synthetic production of the polypeptides of the invention may be according to the solid-phase synthetic method described by Goodman M. (ed.), “Synthesis of Peptides and Peptidomimetics” in Methods of organic chemistry (Houben-Weyl) (Workbench Edition, E22a, b, c, d, e; 2004; Georg Thieme Verlag, Stuttgart, New York)., herein expressly incorporated fully by reference. This technique is well understood and is a common method for preparation of peptides. The general concept of this method depends on attachment of the first amino acid of the chain to a solid polymer by a covalent bond. Succeeding protected amino acids are added, on at a time (stepwise strategy), or in blocks (segment strategy), until the desired sequence is assembled. Finally, the protected peptide is removed from the solid resin support and the protecting groups are cleaved off. By this procedure, reagents and by-products are removed by filtration, thus eliminating the necessity of purifying intermediaries.

Amino acids may be attached to any suitable polymer as a resin. The resin must contain a functional group to which the first protected amino acid can be firmly linked by a covalent bond. Various polymers are suitable for this purpose, such as cellulose, polyvinyl alcohol, polymethylmethacrylate and polystyrene. Suitable resins are commercially available and well known to those of skill in the art. Appropriate protective groups usable in such synthesis include tert-butyloxycarbonyl (BOC), benzyl (Bzl), t-amyloxycarbonyl (Aoc), tosyl (Tos), o-bromo-phenylmethoxycarbonyl (BrZ), 2,6-dichlorobenzyl (BzlCl₂), and phenylmethoxycarbonyl (Z or CBZ). Additional protective groups are identified in Goodman, cited above, as well as in McOmie J F W: Protective Groups in Organic Chemistry, Plenum Press, New York, 1973, both references expressly incorporated fully herein by reference.

General procedures for preparing SAVA compositions of the present invention of this invention involve initially attaching a carboxyl-terminal protected amino acid to the resin. After attachment the resin is filtered, washed and the protecting group on the alpha-amino group of the carboxyl-terminal amino acid is removed. The removal of this protecting group must take place, of course, without breaking the bond between that amino acid and the resin. The next amino, and if necessary, side chain protected amino acid, is then coupled to the free amino group of the amino acid on the resin. This coupling takes place by the formation of an amide bond between the free carboxyl group of the second amino acid and the amino group of the first amino acid attached to the resin. This sequence of events is repeated with successive amino acids until all amino acids are attached to the resin. Finally, the protected peptide is cleaved from the resin and the protecting groups removed to reveal the desired peptide. The cleavage techniques used to separate the peptide from the resin and to remove the protecting groups depend upon the selection of resin and protecting groups and are known to those familiar with the art of peptide synthesis.

Peptides may be cyclized by the formation of a disulfide bond between two cysteine residues. Methods for the formation of such bonds are well known and include such methods as those described in G. A. Grant (Ed.) Synthetic Peptides: A User's Guide 2^{nd} Ed., Oxford University Press, 2002, W. C. Chan and P. D. White (Eds.) Fmoc Solid Phase Synthesis A Practical Approach, Oxford University Press, 2000 and references therein.

Alternative techniques for peptide synthesis are described in Bodanszky et al, Peptide Synthesis, 2nd ed, John Wiley and Sons, New York, 1976, expressly incorporated herein fully by reference. For example, the peptides of the invention may also be synthesized using standard solution peptide synthesis methodologies, involving either stepwise or block coupling of amino acids or peptide fragments using chemical or enzymatic methods of amide bond formation (see, e.g. H. D. Jakubke in The Peptides, Analysis, Synthesis, Biology, Academic Press, New York, 1987, p. 103-165; J. D. Glass, ibid., pp. 167-184; and European Patent 0324659 A2, describing enzymatic peptide synthesis methods.) These solution synthesis methods are well known in the art.

Commercial peptide synthesizers, such as the Applied Biosystems Model 430A, are available for the practice of these methods.

In one aspect of this invention, various forms of a biologically active agent may be used which are capable of being released by the SAVA composition, for example, into adjacent tissues or fluids upon administration to a subject.

In an exemplary embodiment, the compositions of the present invention are used after surgical resection of a tumor in a subject. The compositions are applied to the tissue margins and surrounding tissues after removal of the tumor. The tissues are then surgically closed.

In one embodiment, the removal of tumor tissue may be carried out within the context of any standard surgical process allowing access to and removal of the tumor, including open surgery and laparoscopic techniques. Once the diseased tissue is accessed, and removed, the SAVA composition of the invention is placed in contact with the surrounding tissue along with any surgically acceptable patch or implant, if needed.

In accordance with yet another embodiment, the present invention provides a method of treating a tumor in a subject comprising administering to the mammal in situ, a therapeutically effective amount of the SAVA compositions described above, sufficient to slow, stop or reverse the growth of the tumor in the mammal.

In accordance with an embodiment, the present invention provides a method of local administration of one or more biologically active agents to a subject comprising in situ application of a composition comprising one or more SAVA compositions, described herein, to the site of interest.

As used herein, the term “application” refers to the local in situ administration of the compositions of the present invention to the site of interest. The administration of the compositions of the present invention can be by any known means for contacting the hydrogel with the tissues or tumor of interest. Such means would include, for example, injection, spraying, swabbing, brushing, etc., the SAVA compositions to the tissues.

Without being held to any particular mechanism of action, the compositions of the present invention allow for the sustained release of verteporfin and/or biologically active agents into the surrounding tissues post-operatively to enhance the effectiveness of the surgical treatment by local chemotherapeutic action on any remaining tumor cells, including tumor stem cells, which evaded surgical resection. Verteporfin and/or biologically active agents will be released from the SAVA compositions through dissolution and through the biodegradation of the hydrogel and the bonds between the Pep and V and/or D and linkers, to allow diffusion of V and/or D to come into contact with the surrounding tissues.

An advantage of the compositions and methods described herein is the fact that the use of local administration, allows for high concentrations of V and/or D at the site of the tumor without having systemic effects in the subject.

Another advantage of the compositions and methods described herein is the ability to provide chemotherapy in a sustained release formulation, in parts of the body where there might otherwise be limited access of the biologically active agent to the site of interest. For example, the brain is well known for the blood-brain barrier preventing hydrophobic and polar molecules from entering the brain tissues. Systemic administration causes systemic side effects away from the tumor site. Other tissues in the body have also limited blood flow or circulation, such as bone, kidney, the eye, etc. However, application of the compositions of the present invention directly into these tissues after tumor resection, avoids this common problem.

The dose of the SAVA compositions of the present invention also will be determined by the existence, nature and extent of any adverse side effects that might accompany the administration of a particular composition. Typically, an attending physician will decide the dosage of the pharmaceutical composition with which to treat each individual subject, taking into consideration a variety of factors, such as age, body weight, general health, diet, sex, compound to be administered, route of administration, and the severity of the condition being treated. By way of example, and not intending to limit the invention, the dose of the pharmaceutical compositions of the present invention can be about 0.001 to about 1000 mg/kg body weight of the subject being treated, from about 0.01 to about 100 mg/kg body weight, from about 0.1 mg/kg to about 10 mg/kg, and from about 0.5 mg to about 5 mg/kg body weight. In another embodiment, the dose of the pharmaceutical compositions of the present invention can be at a concentration from about 1 nM to about 10,000 nM, preferably from about 10 nM to about 5,000 nM, more preferably from about 100 nM to about 500 nM.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

In accordance with the embodiments of the present invention, the SAVA compositions for treating a tumor in a subject can encompass many different formulations known in the pharmaceutical arts, including, for example, sustained release formulations. With respect to the inventive methods, the disease can include cancer. Cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor. Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer.

As used herein, the term “proliferative disease” includes cancer and other diseases such as neoplasias and hyperplasias. Cellular proliferative diseases include, for example, rheumatoid arthritis, inflammatory bowel disease, osteoarthritis, leiomyomas, adenomas, lipomas, hemangiomas, fibromas, vascular occlusion, restenosis, artherosclerosis, a pre-neoplastic lesion, carcinoma in situ, oral hairy leukoplakia, or psoriasis. In accordance with one or more embodiments, the term cancer can include, for example cancers of the lung, liver, pancreas, prostate, breast and central nervous system, including glioblastomas and related tumors. In accordance with another embodiment, the term “cancer” includes cancers in tissues that can tolerate high doses of radiation. A high dose of radiation would include doses greater than 2 Gy.

In an embodiment, the cancers treated by the present invention would also include cancers which are resistant to hypoxia, chemotherapy, such as, for example, tamoxifen or taxol resistant cancers, and cancers already resistant to radiation therapy.

In another embodiment, the term “administering” means that at least one or more SAVA compositions of the present invention are introduced into a subject, preferably a subject receiving treatment for a tumor, at the tumor site and surrounding tissues, and the at least one or more compositions are allowed to come in contact with the one or more tumor cells or population of cells.

As used herein, the term “treat,” as well as words stemming therefrom, includes diagnostic and preventative as well as disorder remitative treatment.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

In certain embodiments, the subject compositions comprise about 1% to about 75% or more by weight of the total composition, alternatively about 2.5%, 5%, 10%, 20%, 30%, 40%, 50%, 60% or 70%, of a biologically active agent.

The “therapeutically effective amount” of the pharmaceutical compositions to be administered will be governed by such considerations, and can be the minimum amount necessary to prevent, ameliorate or treat a tumor of interest. As used herein, the term “effective amount” is an equivalent phrase refers to the amount of a therapy (e.g., a prophylactic or therapeutic agent), which is sufficient to reduce the severity and/or duration of a disease, ameliorate one or more symptoms thereof, prevent the growth of a tumor or cause regression of a tumor, or which is sufficient to result in the prevention of the development, recurrence, onset, or progression of a disease or one or more symptoms thereof, or enhance or improve the prophylactic and/or therapeutic effect(s) of another therapy (e.g., another therapeutic agent) useful for treating a disease, such as cancer.

It will be understood by those of skill in the art that the methods for making the SAVA compositions of the present invention can use any known solvents or mixtures thereof that will dissolve the SAVA compositions. Known methods for extraction of the mixtures and drying can also be used.

In accordance with another embodiment, the present invention provides a method of treating cancer in a subject comprising a) administering to the subject an effective amount of the making the SAVA compositions of the present invention, and a pharmaceutically acceptable carrier, in one or more doses, and b) administering ionizing radiation to the subject in proximity to the location of the cancer in the subject.

Radiation therapy, radio-immunotherapy or pre-targeted radioimmunotherapy are used for the treatment of diseases of oncological nature. “Radiotherapy”, or radiation therapy, means the treatment of cancer and other diseases with ionizing radiation. Ionizing radiation deposits energy that injures or destroys cells in the area being treated (the target tissue) by damaging their genetic material, making it impossible for these cells to continue to grow. Radiotherapy may be used to treat localized solid tumors, such as cancers of the skin, tongue, larynx, brain, breast, lung or uterine cervix. It can also be used to treat leukemia and lymphoma, i.e. cancers of the blood-forming cells and lymphatic system, respectively. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons are machines that produce x-rays of increasingly greater energy. The use of machines to focus radiation (such as x-rays) on a cancer site is called external beam radiotherapy. Gamma rays are another form of photons used in radiotherapy. Gamma rays are produced spontaneously as certain elements (such as radium, uranium, and cobalt 60) release radiation as they decompose, or decay. Another technique for delivering radiation to cancer cells is to place radioactive implants directly in a tumor or body cavity. This is called internal radiotherapy. Brachytherapy, interstitial irradiation, and intracavitary irradiation are types of internal radiotherapy. In this treatment, the radiation dose is concentrated in a small area, and the patient stays in the hospital for a few days. Internal radiotherapy is frequently used for cancers of the tongue, uterus, and cervix. A further technique is intra-operative irradiation, in which a large dose of external radiation is directed at the tumor and surrounding tissue during surgery. Another approach is particle beam radiation therapy. This type of therapy differs from photon radiotherapy in that it involves the use of fast-moving subatomic particles to treat localized cancers. Some particles (neutrons, pions, and heavy ions) deposit more energy along the path they take through tissue than do x-rays or gamma rays, thus causing more damage to the cells they hit. This type of radiation is often referred to as high linear energy transfer (high LET) radiation. Radio-sensitizers make the tumor cells more likely to be damaged, and radio-protectors protect normal tissues from the effects of radiation.

Ionizing radiation is widely used for the treatment of solid tumors. Conventional definitive radiation treatment involves multiple treatments, generally 20-40, with low doses (<2-3 Gy) stretching over weeks. Promising evidence indicates that high dose, >15-20 Gy, radiotherapy given in <5 treatments also known as stereotactic ablative radiotherapy (SABR) provides therapeutic benefit to human tumors. The first modern venture into SABR was with the use of stereotactic radiosurgery (SRS) for small intracranial tumors that was made possible by technology allowing for submillimeter delivery precision and steep dose gradients beyond the tumor target. SABR, which is also known as stereotactic body radiation therapy (SBRT) has developed more recently with newer technologic advances to target tumors outside of the brain and includes tumors of practically every major body site. Early clinical experience with SABR in early stage lung cancer and oligometastatic cancer has demonstrated excellent local control of ˜90%. However, the extreme doses used for SABR can be associated with prominent normal tissue toxicity. Thus, because of the technical complexity and increased toxicity with delivery of SABR there has been an ongoing search for tumor selective radiation sensitizers that would enable use of lower dose per fraction. In addition, too little is known regarding the mechanisms by which SABR acts on tumors in vivo to assume that conventional dose radiation sensitizers, such as platinum agents, would also enhance SABR.

As used herein, the term “treatment,” as well as words stemming therefrom, includes, but is not limited to administering one or more doses of radiotherapy to the site of a tumor in a subject or a cell or population of cells, including the use of SABR, SRS and SBRT methods. It will be understood that a subject may undergo more than one treatment or cycle of radiotherapy to be effective in reducing tumor volume or initiate cancer/target cell death. It will be understood that the radiotherapy will be administered locally to the site of the tumor and surrounding tissues, either before, during, or after treatment of the tumor in situ with the SAVA compositions of the present invention.

In accordance with an embodiment, the present invention provides a SAVA composition comprising the compositions described above, and a pharmaceutically acceptable carrier, for use as a medicament, preferably for use as a radiation dose sensitizer in a subject suffering from a proliferative disease and undergoing radiation therapy.

These and other aspects of the present invention will be further appreciated upon consideration of the following Examples, which are intended to illustrate certain particular embodiments of the invention but are not intended to limit its scope, as defined by the claims.

### Example 1

Ver-RGDR was synthesized in two steps. In the first step, the peptide RGDR (SEQ ID NO: 3) was synthesized using AAPPTEC Focus XC synthesizer via standard Fmoc-solid phase technique. Fmoc groups were deprotected using 20% 4-methylpiperidine in DMF, and amino acid/HBTU/DIEA (4/3.98/6) was applied for coupling. In the second step, verteporfin (Ver) was conjugated onto the backbone amino groups of N-terminus arginine (Verteporfin/HBTU/DIEA (4/3.98/6). The finished conjugate was cleaved from the resin with TFA/TIS/water (92.5:5:2.5) solution. The conjugate was confirmed by ESI MS m/z for 401.4 [M+3H], 601.9 [M+2H], 1202.7 [M+H], C₅₉H₇₄N₁₄O₁₄, calcd. 1203.3.

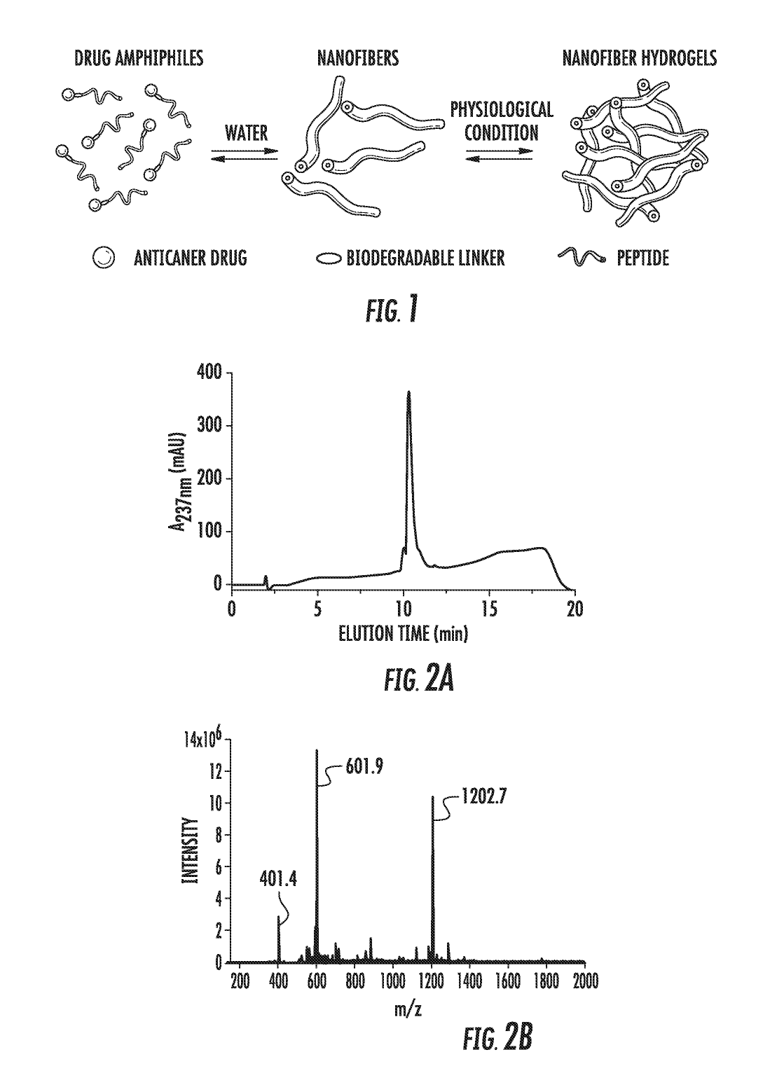

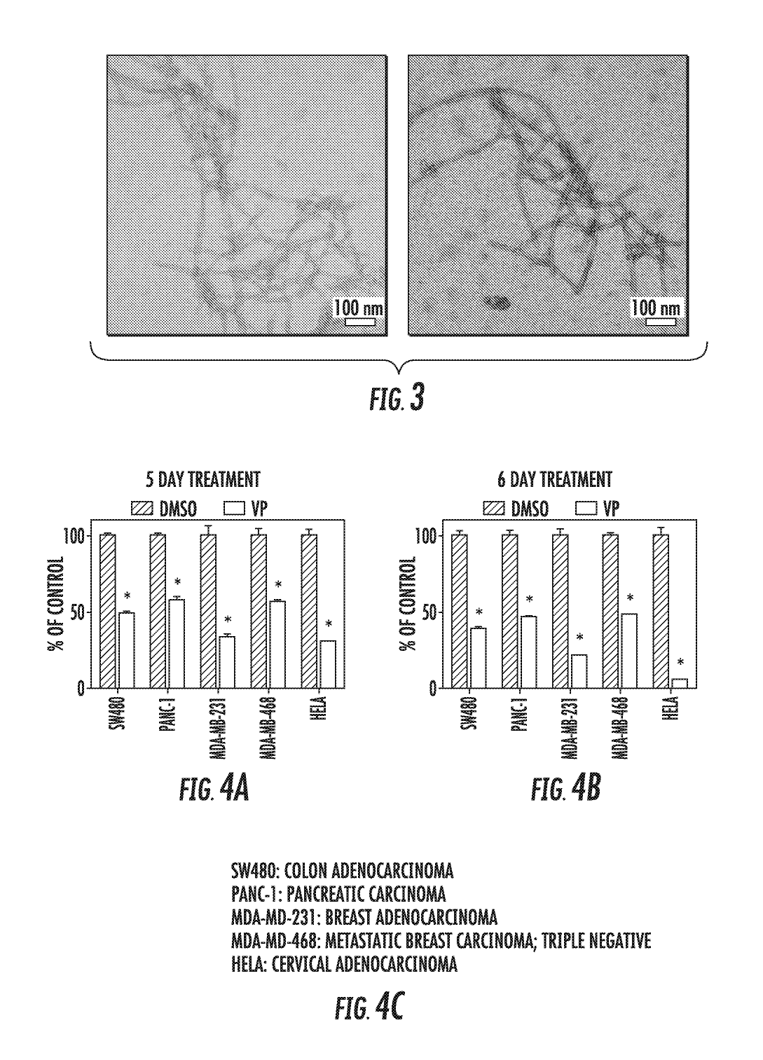

The compound Ver-RGDR was prepared, wherein the verteporfin molecule was linked directly to the RGDR (SEQ ID NO: 1) target (T) moiety via an amide bond. Samples of the compound were run on reverse-phase HPLC and ESI-MS which shows high purity of the compound and the expected molecular mass (FIG. 2). Ver-RGDR was dissolved in DI water at 5 mM and aged for 24 hours. After 24 hours of aging, short fibrous structures were observed in TEM images (FIG. 3).

### Example 2

Ver-Mercapto-RGDR Synthesis.

Synthetic Steps:

Peptide Synthesis of AcCRGDR. The peptide AcCRGDR is synthesized using AAPPTEC Focus XC synthesizer via standard Fmoc-solid phase technique. Fmoc groups were deprotected using 20% 4-methylpiperidine in DMF, and amino acid/HBTU/DIEA (4/3.98/6) was applied for coupling. The N-terminal amine was acetylated manually by reacting with 20% acetic anhydride in DMF. The finished peptide was cleaved from the resin with TFA/TIS/water (92.5:5:2.5) solution.

Verteporfin ester synthesis. 2-Mercaptoethanol and aldrithiol are dissolved into methanol and stirred for 3 hours. The solution is purified by RP-HPLC, leading to product 2-(pyridin-2-yldisulfanyl) ethanol. Verteporfin, 2-(pyridin-2-yldisulfanyl) ethanol, N,N′-diisopropylcarbodiimide and 4-Dimethylaminopyridine are added into an oven dried flask with a stirrer bar, evacuated and refilled with nitrogen three times to remove air, then dissolved in anhydrous acetonitrile. The reaction is allowed to stir in the dark at room temperature for 48 hours. The solvents are removed in vacuo and the residue is dissolved in chloroform and purified by flash chromatography to give the product.

Verteporfin-mercapto-RGDR synthesis. AcCRGDR and verteporfin are added to an oven dried flask equipped with a stirrer bar and evacuated and filled with nitrogen three times to remove the air. The reagents are then dissolved in anhydrous DMF. The solution is allowed to stir for 16 hours, before purification by RP-HPLC. Product fractions are combined and lyophilized to give final product.

When using a hydrazone linker, verteporfin is first conjugated with selected hydrazone, leading to verteporfin prodrug, and then reacted with peptides. When using a short peptide linker, linker is coupled onto peptide using standard Fmoc-solid phase technique, and verteporfin is conjugated onto peptide with HBTU and DIEA.

### Example 3

Treatment of cancer cell lines with Ver-RGDR decreases proliferation of non-CNS tumor cells in vitro.

Cells tested were subjected to the following protocol: Day 1: 7000 cells per well were seeded in a 96-well plate. Day 3: cells were treated with 12.5 μM of Verteporfin (treatment group) and its equivalent DMSO concentration (control group) in 200 μl of media/well. Proliferation was assessed using MTT at Day 8, 9 using the following method:

MTT stock solution (5 mg/ml) is added to each culture being assayed to equal one-tenth the original culture volume and incubated for 4 hr. At the end of the incubation period the medium is removed the converted dye is solubilized with 100% isopropanol. Absorbance of converted dye is measured at a wavelength of 570 nm.

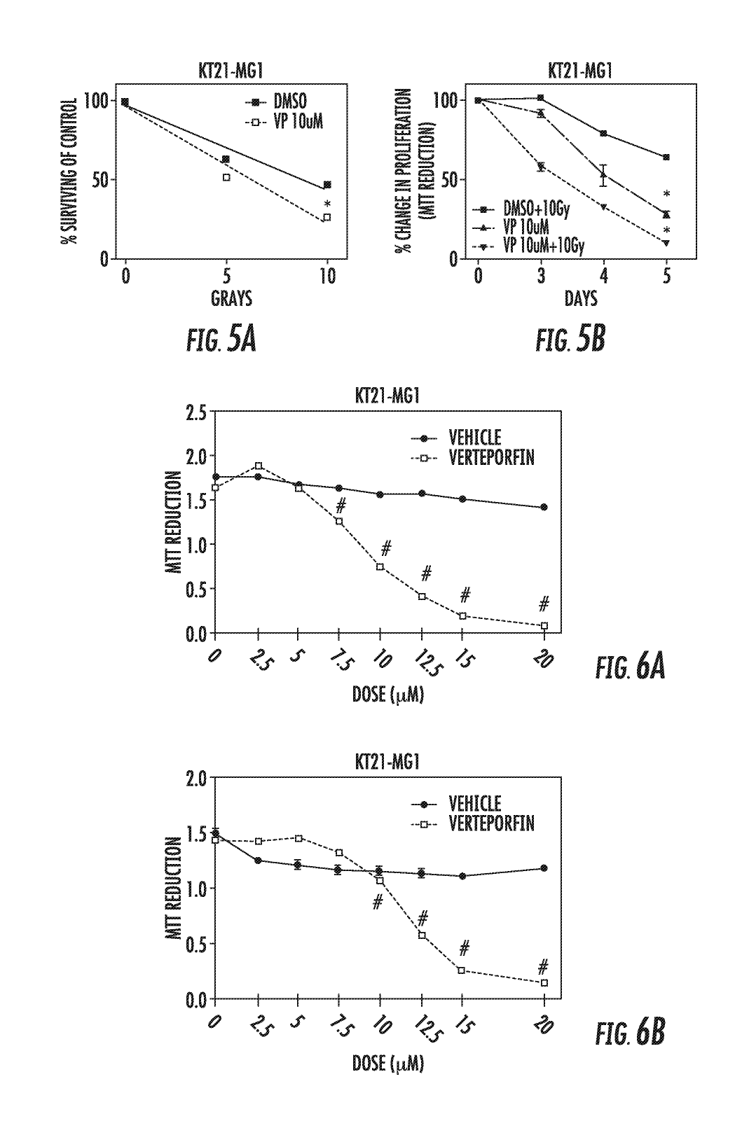

The cell lines SW480 (colon adenocarcinoma), PANC-1 (pancreatic cancer), MDA-MD-231 (breast adenocarcinoma), MDA-MD-468 (metastatic breast carcinoma triple negative), and HELA (cervical carcinoma) were treated with 12.5 μM of the SAVA composition, Ver-RGDR, or DMSO (solvent control) for 5 days (FIG. 5a) or 6 days (FIG. 5b) in culture. Cell proliferation was then assessed using the MTT protocol. Results showed that the SAVA composition reduced proliferation in the treated cell lines by about 50% after 5 days and up to 70% after 6 days in MDA-MD-231 cells and 80% in HELA cell lines. P-values: All cell lines tested at day 5 and 6 had p<0.0001 compared to their corresponding DMSO control. The results were statistically significant.

### Example 4

Pretreatment of tumors with SAVA compositions induces radiosensitivity. Cells from the tumor cell line KT21G1 (meningioma) were pretreated with 10 μM Ver-RGDR, or DMSO (solvent control) for 12 hours in vitro as described in Example 3. Cells were then irradiated with 0, 5 or 10 Gy of radiation and assessed at day 0, 3, 4, and 5 post-irradiation. Cell proliferation was then assessed using the MTT protocol. Results showed that the SAVA composition reduced proliferation in the treated cell lines in a radiation dose specific (FIG. 6a) and time dependent manner (FIG. 6b) compared to controls. The SAVA compositions without irradiation had significant effect of reducing cell proliferation and the effect was increased significantly after 10 Gy of irradiation and 5 days exposure to the SAVA composition. All values with * had p<0.002 compared to their corresponding DMSO control. The results were statistically significant.

### Example 5

Treatment of cancer cell lines with Ver-RGDR decreases proliferation of CNS tumor cells in vitro.

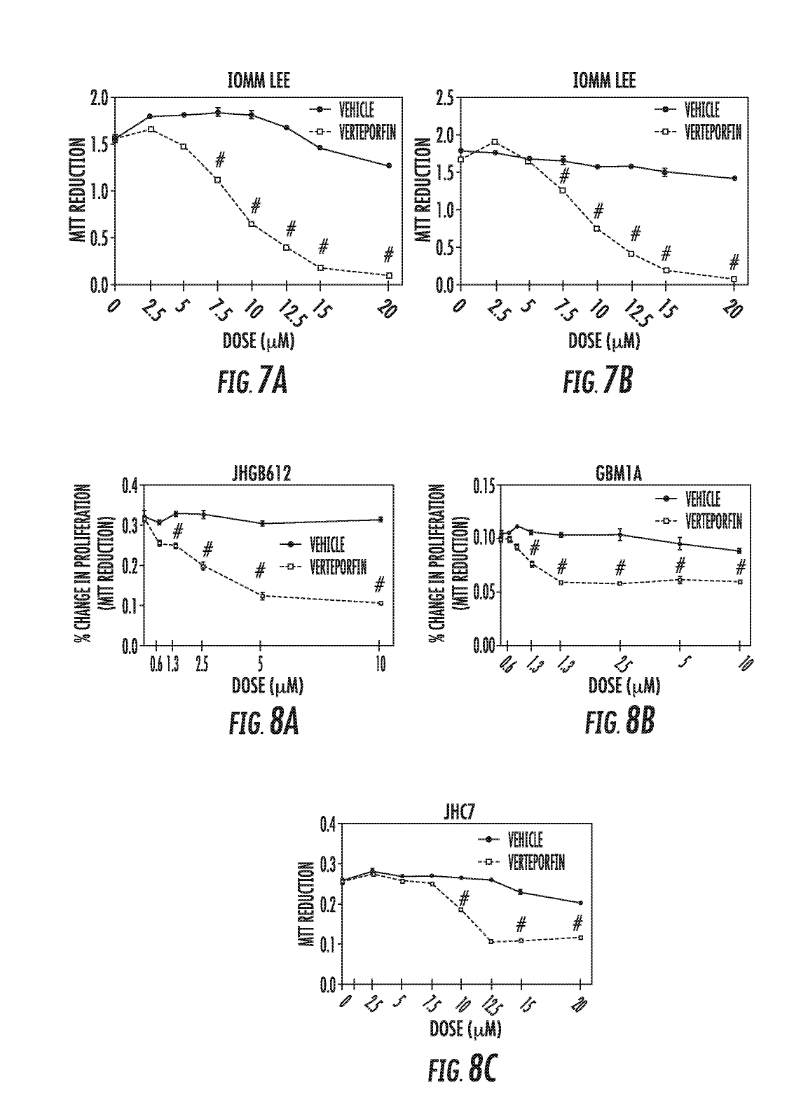

The malignant meningioma cell lines KT21-MG1 (FIG. 7), IOMM-Lee (FIG. 8), the primary patient-derived GBM cell lines, JHGB612 and GBM1A, and a patient-derived primary chordoma cell line, JHC7 (FIG. 9) were all tested with a SAVA composition of the present invention, using the protocol of Example 3. All should significant dose-dependent effect on cell survival when compared to controls.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A self-assembling verteporfin amphiphile composition (SAVA) having the formula:
V-Pep;
wherein V-Pep comprises at least one or more verteporfin molecules (V) conjugated to a hydrophilic peptide composition (Pep);
wherein Pep is a hydrophilic peptide composition having the amino acid sequence L-Bₙ-(T)_{z},
L is a C₂-C₆ alkyl linker having at least one or more disulfide bonds;
Bₙ is an amino acid linker, of n=0 to 12 amino acids, which can be the same or different; and
T is a targeting peptide of z=1 to 15 amino acids.

2. The verteporfin amphiphile composition of claim 1, wherein V is at least one to four, or more verteporfin molecules covalently linked to Pep.

3. The composition of claim 1, wherein B is an amino acid selected from the group consisting of cysteine, methionine, phenylalanine, lysine, valine and tyrosine.

4. The composition of claim 1, wherein n is 1 to 3 amino acids

5. The composition of claim 4, wherein B is cysteine.

6. The composition of claim 1, wherein T is selected from the group consisting of RGD, RGDR (SEQ ID NO: 2), HDK, CEA, TAG-72, CyclinB1, Ep-CAM, Her2/neu, CDK4, fibronectin, p53, and ras.

7. The composition of claim 6, wherein T is RGDS (SEQ ID NO: 1).

8. The composition of claim 1, wherein the composition has the formula of formula I:

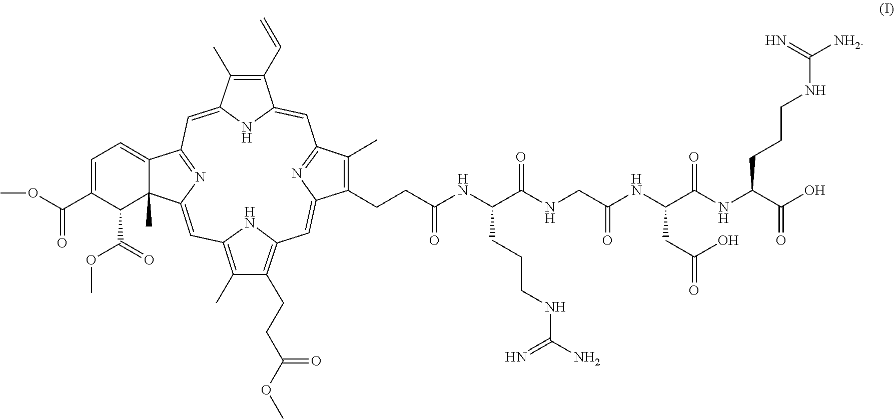 [SMILES: C=Cc1c(C)c2cc3nc(cc4[nH]c(cc5nc(cc1[nH]2)C(C)=C5CCC(=O)N[C@@H](CCCNC(=N)N)C(=O)NCC(=O)N[C@@H](CC(=O)O)C(=O)N[C@@H](CCCNC(=N)N)C(=O)O)c(CCC(=O)OC)c4C)[C@@]1(C)C3=CC=C(C(=O)OC)[C@H]1C(=O)OC]

9. The composition of claim 1, wherein the composition has the formula of formula II:

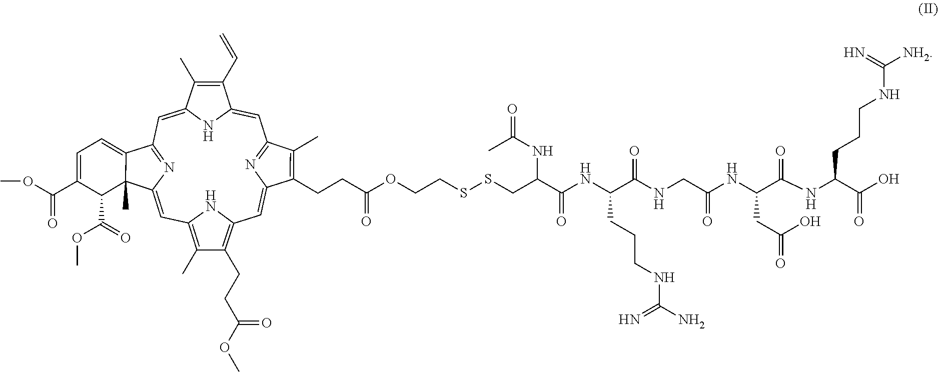 [SMILES: C=Cc1c(C)c2cc3nc(cc4[nH]c(cc5nc(cc1[nH]2)C(C)=C5CCC(=O)OCCSSCC(NC(C)=O)C(=O)N[C@@H](CCCNC(=N)N)C(=O)NCC(=O)N[C@@H](CC(=O)O)C(=O)N[C@@H](CCCNC(=N)N)C(=O)O)c(CCC(=O)OC)c4C)[C@@]1(C)C3=CC=C(C(=O)OC)[C@H]1C(=O)OC]

10. The composition of claim 1, wherein the verteporfin amphiphile composition is covalently linked to an imaging agent.

11. The composition of claim 1, wherein L is 2-mercaptoethanol.

12. A self-assembling verteporfin amphiphile composition comprising the composition of claim 1, and at least one biologically active agent (D) in a mixture.

13. The composition of claim 12, wherein D is a cancer chemotherapeutic drug.

14. The composition of claim 13, wherein D is is an alkylating agent, nitrogen mustard alkylating agent, nitrosourea alkylating agent, antimetabolite, purine analog antimetabolite, pyrimidine analog antimetabolite, hormonal antineoplastic, natural antineoplastic, antibiotic natural antineoplastis, vinca alkaloid natural antineoplastic, carboplatin, cisplatin, carmustine (BCNU), methotrexate, fluorouracil (5-FU), gemcitabine, goserelin, leuprolide, tamoxifen, aldesleukin, interleukin-2, docetaxel, etoposide, interferon, paclitaxel, other taxane derivatives, tretinoin (ATRA), bleomycin, dactinomycin, daunorubicin, doxorubicin, mitomycin, bumetanide, verteporfrin, vorapaxar, and camptothecin.

15. The composition of claim 12, wherein D is at least two different biologically active agents.

16. A method for treating a tumor in a subject comprising administering to the subject at the site of the tumor, an effective amount of the composition of claim 1.

17. The method of claim 16, further comprising surgically removing the tumor from the selected tissue of the subject prior to, or concurrently with, administration of an effective amount of the composition of claim 1 to the subject.

18. The method of claim 17, wherein the selected tissue of the subject is lung, breast, colon, prostate, liver, pancreas, brain and cervical.

19. A method of treating cancer in a subject comprising a) administering to the subject an effective amount of the making the SAVA compositions of claim 1, and a pharmaceutically acceptable carrier, in one or more doses, and b) administering ionizing radiation to the subject in proximity to the location of the cancer in the subject.

20. The method of claim 19, wherein the ionizing radiation dose is in the range of 0.1 Gy to about 30 Gy, preferably in a range of 5 Gy to about 25 Gy.

21. The method of claim 20, wherein the radiation is sterotactic ablative radiotherapy (SABR) or sterotactic body radiation therapy (SBRT).


SEQUENCE LISTING (1 sequence)

SEQ ID NO: 1 - protein, 4 aa, Artificial Sequence
  synthetic sequence
RGDR